(19) United States
(12) Patent Application Publication
(10) Pub. No.: US 2003/0185794 A1
(43) Pub. Date: Oct. 2, 2003

(54) Therapeutic angiogenic factors and methods for their use

(76) Inventor: Kenneth J. Colley, San Mateo, CA (US)

Correspondence Address: Jill A. Jacobson, Morrison & Foerster LLP, 755 Page Mill Road, Palo Alto, CA, 94304-1018, US

(21) Appl. No.: 10/323,533

(22) Filed: Dec. 18, 2002

Related U.S. Application Data
(62) Division of application No. 09/293,287, filed on Apr. 16, 1999.
(60) Provisional application No. 60/082,155, filed on Apr. 17, 1998.

(51) Int. Cl. A61K 38/19; A61K 38/17

(52) U.S. Cl. 424/85.1; 514/12

(57) ABSTRACT

Methods are provided for stimulating angiogenesis in a human or animal in need thereof. Also provided are compositions comprising an angiogenic factor in a pharmaceutically acceptable carrier. In one embodiment, the method comprises administering to the human or other animal a therapeutically effective amount of an angiogenic factor, such as a pleiotrophin or midkine protein, in a pharmaceutically acceptable carrier. The carrier in one embodiment comprises a controlled release matrix, such as a polymer, that permits controlled release of the angiogenic factor. The polymer may be biodegradable and/or bioerodible and preferably biocompatible. Polymers which may be used for controlled release include, for example, poly(esters), poly(anhydrides), and poly(amino acids). Exemplary polymers include silk elastin poly(amino acid) block copolymers and poly-lactide-co-glycolide. In a further embodiment, the angiogenic factor may be provided in a carrier comprising a liposome, such as a heterovesicular liposome. The carrier, such as a liposome, may be provided with a targeting ligand capable of targeting the carrier to a preselected site in the body. The angiogenic factor may be administered to the vascular system, for example the cardiovascular system, or the peripheral vascular system. In a preferred embodiment, the angiogenic factor is a pleiotrophin protein, or a midkine protein. In another embodiment, a method is provided for stimulating angiogenesis in a human or animal comprising administering a therapeutically effective amount of a gene transfer vector encoding the production of pleiotrophin or midkine protein in a pharmaceutically acceptable carrier. The gene transfer vector may be, for example, naked DNA or a viral vector, and may be administered, for example, in combination with liposomes.

### CROSS REFERENCE TO RELATED APPLICATIONS

This application claims the benefit of U.S. Provisional Patent Application Serial No. 60/082,155, filed Apr. 17, 1998, the disclosure of which is incorporated herein by reference in its entirety.

### TECHNICAL FIELD

This invention relates generally to the use of therapeutic angiogenic factors, such as pleiotrophin, to promote angiogenesis for the treatment of a variety of indications including cardiovascular diseases.

### BACKGROUND ART

Polypeptide growth factors have been shown to play important physiological roles in the timely development of tissues during embryonal and neonatal growth and, therefore, their expression is tightly regulated. Conversely, polypeptide growth factor gene expression is deregulated in tumor cell lines, as well as in solid tumors. Cross and Dexter, Cell, 64:271 (1991).

Pleiotrophin (PTN) is a secreted growth factor that belongs to a family of heparin binding growth factors. Lai et al., Biochem. Biophys. Res. Commun., 187:1113-1121 (1992). Pleiotrophin originally was purified as a weak mitogen from bovine uterus and as a neurite outgrowth promoter from neonatal rat brain. Milner et al., Biochem. Biophys. Res. Commun., 165:1096-1103 (1989); Rauvala, EMBO J., 8:2933-2941 (1989); and Li et al., Science, 250:1690-1694 (1990). The purification of an 18-kDa heparin-binding growth factor from the conditioned media of a human breast cancer cell line has been reported. Wellstein et al., J. Biol. Chem., 267:2582-2587 (1992). The cDNAs for human, bovine and rat PTNs have been cloned and sequenced. Fang et al., J. Biol. Chem., 267:25889-25897 (1992); Li et al. (1990) supra; Lai et al. (1992), supra; Kadomatsu et al., Biochem. Biophys. Res. Commun., 151:1312-1318 (1988); Tomomura et al., J. Biol. Chem., 265:10765-10770 (1990); Vrios et al., Biochem. Biophys. Res. Commun., 175:617-624 (1991); and Li et al., J. Biol. Chem., 267:26011-26016 (1992).

PTN belongs to a family of heparin-binding proteins which include the midkine (MK) growth factor proteins. Midkine protein has approximately 50% amino acid homology to PTN. Kadomatsu et al., J. Cell. Biol., 110:607-616 (1990); and Kretschmer et al., Growth Factors 5:99-114 (1991). PTN and the MK proteins appear to play a role during development of the neuroectoderm. The physiologic expression of the genes in the adult occurs only in very restricted areas of the nervous system. Böhlen and Kovesdi, Prog. Growth Factor Res., 3:143-157 (1991).

PTN acts as a growth factor in tumors. Antisense nucleotides to PTN have been developed to inhibit tumor formation, as described in PCT WO 96/02257, the disclosure of which is incorporated herein. Expression of PTN is elevated in melanomas that are highly vascularized, and PTN supports the growth of SW13 cells in soft agar. Wellstein et al., J. Biol. Chem. 267:2582-2587 (1992). PTN purified from different sources has been described as having mitogenic activity for endothelial and epithelial cells and fibroblasts. See, e.g. Fang et al., J. Biol. Chem., 267:25889-25897 (1992); Kuo et al., J. Biol. Chem., 265:18749-18752 (1990); Rauvala, EMBO J., 8:2933-2941 (1989); Merenmies and Rauvala, J. Biol. Chem., 265:16721-16724 (1990); Li et al., Science, 250:1690-1694 (1990); and Milner et al., Biochem. Biophys. Res. Commun., 165:1096-1103 (1989)). PTN has shown mitogenic activity for bovine brain capillary cells and angiogenic activity in the rabbit cornea assay (Courty et al., Biochem. Biophys. Res. Commun., 180:145-151 (1991)). PTN also has been shown to induce tube formation of endothelial cells in vitro. Laaroubi et al., Growth Factors, 10:89-98 (1994).

PTN mRNA has been detected in human breast cancer samples and in human breast cancer cell lines. Fang et al., J. Biol. Chem., 267:25889-25897 (1992). PTN was also detected in carcinogen-induced rat mammary tumors. Koyama et al., J. Natl. Cancer Inst. 48:1671-1680(1972). Other primary human cancers and cell lines were also found to express PTN, including melanoma, squamous cell carcinomas of the head and neck, neuroblastomas and glioblastomas. PTN appears to be very tightly regulated in the non-cancerous state, expressed only in regions of the brain and reproductive tract, based on rodent models. Bloch et al., Brain Res. Dev. Brain Res., 70:267-278 (1992); and Vanderwinden et al., Anat. Embryol., (Berl) 186:387-406 (1992).

PTN was found to be much more widely expressed during embryonic development, in contrast to the adult. It has been detected in brain, mesenchyme of lung, gut, kidney and reproductive tract, and in bone and cartilage progenitors (Bloch et al., Brain Res. Dev. Brain Res., 70:267-278 (1992); and Vanderwinden et al., Anat. Embryol., (Berl) 186:387-406 (1992)). This suggests an important physiologic role for PTN during brain development and organogenesis.

PTN has been described as pleiotrophin. See, e.g., PCT WO 96/02257, the disclosure of which is incorporated herein. It has been described by different names depending on the tissue source: heparin-affinity regulatory protein, HARP (Courty et al., J. Cell. Biochem., 15F:Abstr. 221-Abstr. 220 (Abstract) (1991); and Biochem. Biophys. Res. Commun., 180:145-151(1991)), heparin-binding neurotrophic factor, HBNF (Kovesdi et al., Biochem. Biophys. Commun., 172:850-854 (1990) and Huber et al., Neurochem. Res., 15:435-439 (1990)) and p18 (Kuo et al., J. Biol. Chem., 265:18749-18752 (1990)) from bovine brain; heparin-binding growth associated molecule, HB-GAM (Rauvala, EMBO J. 8:2933-2941 (1989); and Merenmies and Rauvala, J. Biol. Chem., 265:16721-16724 (1990)) from rat brain; heparin-binding growth factor 8, HBGF-8 (Milner et al., Biochem. Biophys. Res. Commun., 165:1096-1103 (1989)), osteoblast specific factor, OSF-1 (Tezuka et al., Biochem. Biophys. Res. Commun., 173:246-251 (1990)) and pleiotrophin, PTN (Li et al., Science 250:1690-1694 (1990)) from human placenta and rat brain.

The protein structure of PTN has been reported as containing five disulfide bridges which determine its three dimensional structure. The presence of the disulfide bridges result in certain characteristics of the protein, such as its resistance to low pH and sensitivity to reducing conditions. Wellstein et al., J. Biol. Chem., 267:2582-2587 (1992); and Fang et al., J. Biol. Chem., 267:25889-25897 (1992).

There is a need for the development of methods for administering angiogenic growth factors, such as pleiotrophin, in therapeutically effective amounts to patients in need of angiogenic therapy. There is a particular need for the development of therapeutic methods for the use of angiogenic growth factors in the treatment of ischemic conditions. There also is a need for the development of methods for treating vascular diseases such as cardiovascular diseases. There further is a need for delivery systems for delivering angiogenic growth factors, which permit controlled delivery and release of the growth factors.

### DISCLOSURE OF THE INVENTION

Methods are provided for stimulating angiogenesis in a human or animal in need thereof. Also provided are compositions comprising an angiogenic factor in a pharmaceutically acceptable carrier. In one embodiment, the method comprises administering to a human or animal in need thereof a therapeutically effective amount of an angiogenic factor, such as a pleiotrophin or midkine molecule, optionally in a pharmaceutically acceptable carrier. The angiogenic factor may be, for example, a pleiotrophin or midkine protein.

The carrier in one embodiment comprises a controlled release matrix, such as a polymer, that permits controlled release of the angiogenic factor. The polymer may be biodegradable or bioerodable and biocompatible. Polymers which may be used for controlled release include, for example, poly(esters), poly(anhydrides), and poly(amino acids). Exemplary poly(amino acids) include silk elastin poly(amino acid) block copolymers. In a further embodiment, the angiogenic factor may be provided in a carrier comprising a liposome, such as a heterovesicular liposome. The carrier, such as a liposome, may be provided with a targeting ligand capable of targeting the liposome to a preselected site in the body.

In one embodiment, the angiogenic factor is administered to the vascular system, for example, the cardiovascular system, or the peripheral vascular system. The angiogenic factor may be administered in a therapeutically effective amount for the treatment of, for example, coronary artery disease, ischemic heart disease, diabetic peripheral vasculopathies or peripheral atherosclerotic disease. In another embodiment, the angiogenic factor is administered locally in a therapeutically effective amount to a wound to promote wound healing. Wounds that may be treated include ulcers, pressure sores, surgically induced wounds, and traumatically induced wounds.

In a further embodiment, the angiogenic factor is administered locally in a therapeutically effective amount to tissue comprising nerves to treat a neurological condition, such as stroke, multi-infarct dementia, and general brain ischemia. The angiogenic factor further may be administered locally in a therapeutically effective amount to tissue comprising bone or cartilage, for example, for the treatment of conditions such as osteoporosis, arthritis and joint replacement or repair. The angiogenic factor further may be administered locally in a host in a therapeutically effective amount to an organ transplant site to promote engraftment of the transplant in the host.

In a preferred embodiment, the angiogenic factor is a pleiotrophin protein, or a midkine protein, for example, isolated from a human cell source, or an active fragment or analogue thereof, which may be, for example, produced recombinantly in a eukaryotic host cell.

In one embodiment, there is provided a method of stimulating angiogenesis in a human or animal in need thereof, the method comprising administering to the human or animal a therapeutically effective amount of an angiogenic factor in a pharmaceutically acceptable carrier comprising a silk elastin poly(amino acid) block copolymer, and/or a poly-lactide-co-glycolide.

Angiogenic factors which may be used include pleiotrophin, midkine, fibroblast growth factor (FGF) family members, vascular endothelial growth factor (VEGF) family members, platelet derived growth factor (PDGF) family members, and epithelial growth factor (EGF) family members, as well as active fragments and analogues thereof.

In a further embodiment, a method is provided for stimulating angiogenesis in a human or animal in need thereof, the method comprising administering to the human or other animal a therapeutically effective amount of a gene transfer vector encoding the production of a pleiotrophin or midkine protein optionally in a pharmaceutically acceptable carrier. The gene transfer vector may be, for example, naked DNA or a viral vector, and may be administered, for example, in combination with liposomes.

### BRIEF DESCRIPTION OF THE DRAWINGS

FIG. 1 is a graph showing the percent increase in proliferation of endothelial cells over time after treatment with pleiotrophin.

FIG. 2 is a graph showing aggregate vessel cross sectional area over time after treatment of a mouse wound with an implant comprising pleiotrophin.

### MODES FOR CARRYING OUT THE INVENTION

Provided are compositions including angiogenic factors, as well as methods for their manufacture and use. The angiogenic factors may be administered to tissue to revascularize the tissue, for example in the case of damaged or diseased vascular tissue. In one embodiment, the angiogenic factor is provided in a delivery matrix for controlled release of the factor locally at the site of the damage or disease. The methods and compositions promote angiogenesis, the formation of new blood vessels, and thus may be used in a variety of therapeutic applications. Angiogenic factors preferably stimulate the growth of endothelial cells, epithelial cells and fibroblasts at the site of administration. The therapeutic administration of such angiogenic factors to various poorly vascularized tissues can augment the blood supply by stimulating the formation of new blood vessels. Methods and compositions also are provided for delivery of nucleic acid constructs which direct the expression of angiogenic factors.

Angiogenic Factors

As used herein the phrase “angiogenic factor” refers to a molecule that is capable of stimulating angiogenesis. Angiogenic factors include naturally occurring polypeptide growth factors, or biologically active fragments or derivatives or analogues thereof. Angiogenesis is defined as the development of new blood vessels. Angiogenesis in vivo generally involves the stimulation and growth of endothelial cells. In addition, the stimulation of fibroblasts and epithelial cells aids in forming the entire cell population comprising normal vascular tissue, including the outer connective tissue layer of vessels. Folkman, 1992, EXS 61:4-13 and Bicknell et al., 1996, Curr. Opin. Oncol. 8(1):60-65.

In one embodiment, the angiogenic factor is a pleiotrophin molecule. Pleiotrophin molecules include pleiotrophin proteins. The pleiotrophin molecules may be, for example, naturally occurring pleiotrophin proteins, as well as biologically active fragments thereof, and modified and synthetic forms thereof including derivatives, analogs and mimetics, such as small molecule mimetics. Naturally occurring pleiotrophin proteins include proteins of the pleiotrophin family, particularly human pleiotrophin.

Pleiotrophin proteins advantageously can stimulate the proliferation of endothelial cells, epithelial cells and fibroblasts. Pleiotrophin proteins thus advantageously can stimulate both neoangiogenesis and fibroplasia, which are important for natural wound healing and tissue repair. Neoangiogenesis is especially critical to the salvage of ischemic tissues. Pleiotrophin proteins in one embodiment may be isolated from natural sources or by recombinant production. In one embodiment, pleiotrophin is the mature peptide having the sequence encoded by bases 477-980 of SEQ ID NO 1, as described in PCT WO 96/02257, the disclosure of which is incorporated herein.

Other angiogenic factors which are useful include growth factors, such as midkines, members of the vascular endothelial growth factor (VEGF) family, including VEGF-2, VEGF-C and VEGF-D (Plate et al., J. Neurooncol. 35:365-372 (1997); Joukov et al., J. Cell Physiol., 173:211-215 (1997); members of the fibroblast growth factor (FGF) family, including FGF-1 through FGF-18, particularly FGF-1, FGF-2 and FGF-5; hepatoma-derived growth factor (HDGF); hepatocyte growth factor/scatter factor (HGF, Boroset et al., Lancet, 345:293-295 (1995)); members of the epidermal growth factor (EGF) family, including transforming growth factor alpha (TGF-α), EGF, and TGF-α-HIII (Brown, Eur J. Gastroenterol. Hepatol., 7:914-922 (1995) and International Patent Application No. WO 97/25349); and platelet derived growth factors (PDGFs), including AA, AB and BB isoforms (Hart et al., Genet. Eng. 17:181:208 (1995)).

Other angiogenic factors include angiopoictins, such as Ang1, and integrin stimulating factors, for example, Del-1. Ang1 is described in Suri et al., Cell, 87:1171-80 (1996); and Del-1 is described in Hidai et al, Genes Dev., 12:21-33 (1998), the disclosures of each of which are disclosed herein by reference.

In one embodiment, the angiogenic factor is a midkine molecule. Midkine molecules include midkine proteins. The midkine molecules may be, for example, naturally occurring midkine proteins, as well as biologically active fragments thereof, and modified and synthetic forms thereof including derivatives, analogs and mimetics.

The terms “protein”, “polypeptide”, and “peptide” are used interchangeably herein to refer to polymers of amino acids of any length. The polymer may be linear or branched, it may comprise modified amino acids, and it may be interrupted by non-amino acids. It also may be modified naturally or by intervention; for example, disulfide bond formation, glycosylation, myristylation, acetylation, alkylation, phosphorylation or dephosphorylation. Also included within the definition are polypeptides containing one or more analogs of an amino acid (including, for example, unnatural amino acids) as well as other modifications known in the art.

Fibroblast growth factors (FGFs) are generally between 10-20 kDa in molecular mass, although forms of higher mass have been isolated from natural sources. Wilkie et al., Curr. Biol., 5:500-507 (1995). At least 18 members of the FGF family are known (FGF-1 through FGF-18), although the human homologue has not been cloned for all FGF family members. Glycosylation is not required for bioactivity, so proteins from this family may be recombinantly produced in both eukaryotic and prokaryotic expression systems.

It is preferred that the source of the growth factor used match the patient to whom the growth factor is administered (e.g., human pleiotrophin is administered to a human subject). It will be understood by one of skill in the art that the term “source” as used in this context refers to the tissue source of the protein if it is isolated from natural sources, or the source of the amino acid sequence, if the protein is recombinantly produced.

Most angiogenic factors are known to be produced in a number of different “splice variants”. Splice variants are produced by differential splicing of one or more exons from the gene. Not all exons in a gene may be retained in the spliced mRNA that is translated. Variations in mRNA splicing may be specific to developmental stages, particular tissues, or to pathogenic conditions and can lead to the production of a large number of different proteins from the same gene. The angiogenic factors useful in the instant invention include splice variants.

Indications

A variety of indications may be treated using the methods and compositions disclosed herein. Examples include vascular diseases, such as peripheral vascular disease (PVD), including post-surgical or traumatic PVD, and cardiovascular diseases, such as coronary artery disease (CAD). Other vascular diseases which may be treated include diabetic peripheral microangiopathy and other vasculopathies, and claudication due to atherosclerotic disease. Ischemic heart disease states may be treated including inoperable states, such as when there are significant comorbidities. Examples of comorbidities include pulmonary disease, e.g., chronic obstructive pulmonary disease, fragile cardiac condition and arrythmias. Other “inoperable” states which may be treated include patients with intolerance to anestheia, allergies, or who are under combination drug therapy. Stable or unstable new onset angina may be treated. Treatment may be given as adjunct to interventional cardiovascular procedures, such as coronary artery bypass graft and percutaneous transluminal coronary angioplasty (balloon angioplasty). Treatment also may be conducted after failed or restenosed intervention.

The methods and compositions disclosed herein may be used in a variety of applications for wound healing and the treatment of burns. Wound healing applications include chronic cutaneous ulcers, bed or pressure sores, burns, and non-healing wounds. Wounds caused by trauma, such as by accident or by surgery may be treated.

Healing impaired or non-healing wounds may be treated, including non-granulating wounds. For example, wounds associated with diabetes may be treated such as diabetic ulcers. Wounds occurring in immunosuppressed or immunocompromised patients may be treated, for example, in patients undergoing cancer chemotherapy, patients with acquired immunodeficiency syndrome (AIDS), transplant patients, and any patients suffering from medication-induced impaired wound healing.

Other applications include vascularizing regions of tissue that have been cut off from blood supply secondary to resective surgery or trauma, including general surgery, plastic surgery, and transplant surgery, or the treatment of pre-gangrenous ischemic tissue or limb rescue.

The methods and compositions disclosed herein may be used both as a first line therapy, and additionally are useful when other available therapies have been exhausted. Advantageously, patients may be treated who are judged “inoperable” by their physicians, due to surgical risk due to poor general health, or the diffuse nature of their disease wherein they have many small but serious lesions spread throughout the coronary blood supply, rather than one or more main lesions to bypass or open, or others who have undergone failed previous attempts at correcting their disease with invasive procedures.

The methods and compositions described herein may be used in a variety of neurology and neurosurgery applications, for example, for cerebrovascular diseases, such as chronic vascular insufficiency in the brain, multi-infarct dementia (MID), stroke, and general brain ischemia.

Other applications include tissue repair and fortification, and bone repair, including the treatment of osteoporosis, cartilage repair, treatment of arthritis, and joint replacement or repair, as well as hair follicle targeting and treatment of hair loss. Generally, the compositions disclosed herein may be designed for application to a range of injured internal and external tissue, including skin, the reproductive system, the genitourinary system, the pulmonary system, to promote revascularization and endothelial repair. In one embodiment, the compositions may be used in skin repair and cosmetic surgery.

Carriers

The angiogenic factor, such as a pleiotrophin molecule, may be provided in a pharmaceutically acceptable carrier. The carrier may be a biocompatible delivery matrix which permits controlled release of the angiogenic factor in situ. Preferred are matrices in which the angiogenic factor may be incorporated in a stable form while substantially maintaining its activity, and matrices which are biocompatible. Depending upon the selection of the delivery matrix, and the indication being treated, controlled release may be designed to occur on the order of hours, days, weeks, or longer.

The use of a controlled delivery matrix for angiogenic factors, and in particular for pleiotrophin or midkine proteins, has many advantages. Controlled release permits dosages to be administered over time, with controlled release kinetics. In some instances, delivery of the angiogenic factor needs to be continuous to the site where angiogenesis is needed, for example, over several weeks. Controlled release over time, for example, over several days or weeks, or longer, permits continuous delivery of the angiogenic factor to obtain optimal angiogenesis in a therapeutic treatment. The controlled delivery matrix also is advantageous because it protects the angiogenic factor from degradation in vivo in body fluids and tissue, for example, by proteases.

Controlled release from the delivery matrix may designed, based on factors such as choice of carrier, to occur over time, for example, for greater than about 12 or 24 hours. The time of release may be selected, for example, to occur over a time period of about 12 hours to 24 hours; about 12 hours to 42 hours; or, e.g., about 12 to 72 hours. In another embodiment, release may occur for example on the order of about 2 to 90 days, for example, about 3 to 60 days. In one embodiment, the angiogenic factor, such as a pleiotrophin molecule, is delivered locally over a time period of about 7-21 days, or about 3 to 10 days. In the case of a pleiotrophin protein, in one embodiment, the protein is administered over 1, 2, 3 or more weeks in a controlled dosage. The controlled release time may be selected based on the condition treated. For example, longer times may be more effective for wound healing, whereas shorter delivery times may be more useful for some cardiovascular applications.

Controlled release of the angiogenic factor, such as a pleiotrophin protein, from the matrix in vivo may occur, for example, in the amount of about 1 ng to 1 mg/day, for example, about 50 ng to 500 μg/day, or, in one embodiment, about 100 ng/day. Delivery systems comprising the angiogenic factor and the carrier may be formulated that include, for example, 10 ng to 1 mg angiogenic factor, or in another embodiment, about 1 μg to 500 μg, or, for example, about 10 μg to 100 μg, depending on the therapeutic application.

The delivery matrix may be, for example, a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992, the disclosures of which are incorporated herein. The matrix may be, for example, a biodegradable material that can degrade spontaneously in situ and in vivo for example, by hydrolysis or enzymatic cleavage, e.g., by proteases. Optionally, a conjugate of the angiogenic factor and a polymeric carrier may be used.

The delivery matrix may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

Polyesters include poly(α-hydroxyacids) such as poly(lactic acid) and poly(glycolic acid) and copolymers thereof, as well as poly(caprolactone) polymers and copolymers. In a preferred embodiment the controlled release matrix is a poly-lactide-co-glycolide. Controlled release using poly(lactide) and poly(glycolide) copolymers is described in Lewis, “Controlled Release of Bioactive Agents from Lactide/Glycolide Polymers” in “Biodegradable Polymers as Drug Delivery Systems”, Chasin and Langer, eds., Marcel Dekker, New York, 1990, pp. 1-41, the disclosure of which is incorporated herein. Poly-lactide-co-glycolides may be obtained or formed in various polymer and copolymer ratios, for example, 100% D,L-lactide; 85:15 D,L-lactide:glycolide; 50:50 D,L-lactide:glycolide; and 100% glycolide, as described, for example, in Lambert and Peck, J. Controlled Release, 33:189-195 (1995); and Shively et al., J. Controlled Release, 33:237-243 (1995), the disclosures of which are incorporated herein. The polymers can be processed by methods such as melt extrusion, injection molding, solvent casting or solvent evaporation.

The use of polyanhydrides as a controlled release matrix, and the formation of microspheres by hot-melt and solvent removal techniques is described in Chasin et al., “Polyanhydrides as Drug Delivery Systems,” in “Biodegradable Polymers as Drug Delivery Systems”, Chasin and Langer, Eds., Marcel Dekker, New York, 1990, pp. 42-70, the disclosure of which is incorporated herein.

A variety of polyphosphazenes may be used which are available in the art, as described, for example in: Allcock, H. R., “Polyphosphazenes as New Biomedical and Bioactive Materials,” in “Biodegradable Polymers as Drug Delivery Systems”, Chasin and Langer, eds., Marcel Dekker, New York, 1990, pp. 163-193, the disclosure of which is incorporated herein.

Polyamides, such as poly(amino acids) may be used. In one embodiment, the polymer may be a poly(amino acid) block copolymer. For example, fibrin-elastin and fibrin-collagen polymers, as well as other proteinaceous polymers, including chitin, alginate and gelatin may be used. In one embodiment, a silk elastin poly(amino acid) block copolymer may be used. Genetic and protein engineering techniques have been developed which permit the design of silk elastin poly(amino acid) block copolymers with controlled chemical and physical properties. These protein polymers can be designed with silk-like crystalline amino acid sequence blocks and elastin-like flexible amino acid sequence blocks. The properties of these materials are due to the presence of short repeating oligopeptide sequences which may be derived from naturally occurring proteins, such as fibroin and elastin. Exemplary recombinant silk elastin poly(amino acid) block copolymers are described in U.S. Pat. Nos. 5,496,712, 5,514,581, and 5,641,648 to Protein Polymer Technologies; Cappello, J. et al., Biotechnol. Prog., 6:198-202 (1990); Cappello, J., Trends Biotechnol., 8:309-11 (1990); and Cappello et al., Biopolymers, 34:1049-1058 (1994), the disclosures of each of which are incorporated herein by reference.

Poly(phosphoesters) may be used as the controlled delivery matrix. Poly(phosphoesters) with different side chains and methods for making and processing them are described in Kadiyala et al., “Poly(phosphoesters): Synthesis, Physiochemical Characterization and Biological Response,” in “Biomedical Applications of Synthetic Biodegradable Polymers”, J. Hollinger, Ed., CRC Press, Boca Raton, 1995, pp. 33-57, the disclosure of which is incorporated herein.

Polyurethane materials may be used, including, for example, polyurethane amide segmented block copolymers, which are described, for example, in U.S. Pat. No. 5,100,992 to Biomedical Polymers International, the disclosure of which is incorporated herein. Poloxamer polymers may be used, which are polyoxyalkylene block copolymers, such as ethylene oxide propylene oxide block copolymers, for example, the Pluronic gels.

In another embodiment the controlled delivery matrix may be a liposome. Amphiphilic molecules such as lipid containing molecules may be used to form liposomes, as described in Lasic, “Liposomes in Gene Delivery,” CRC Press, New York, 1994, pp. 67-112, the disclosure of which is incorporated herein. Exemplary lipids include lecithins, sphingomyelins, and phosphatidylethanolamines, phosphatidylserines, phosphatidylglycerols and phosphatidylinositols. Natural or synthetic lipids may be used. For example, the synthetic lipid molecules used to form the liposomes may include lipid chains such as dimyristoyl, dipalmitoyl, distearoyl, dioleoyl and palmitoyl-oleoyl chains. Cholesterol may be included. Liposomes and methods for their formation also are described in Nassander, “Liposomes” in “Biodegradable Polymers as Drug Delivery-Systems”, Chasin and Langer, Eds., Marcel Dekker, New York, 1990, pp. 261-338, the disclosure of which is incorporated herein. In one preferred embodiment, a heterovesicular liposome, that includes separate chambers of defined size and distribution may be used, as described, for example in U.S. Pat. Nos. 5,422,120 and 5,576,017 to DepoTech Corporation, the disclosures of which are incorporated herein.

Collagen, albumin, and fibrinogen containing materials may be used, as described, for example, in Bogdansky, “Natural Polymers as Drug Delivery Systems”, in “Biodegradable Polymers as Drug Delivery Systems”, Chasin and Langer, Eds., Marcel Dekker, New York, 1990, pp. 231-259, the disclosure of which is incorporated herein. Exemplary collagen compositions which may be used include collagen-polymer conjugates, as described in U.S. Pat. Nos. 5,523,348, 5,510,418, 5,475,052 and 5,446,091 to Collagen Corporation, the disclosures of which are incorporated herein. Crosslinkable modified collagen including free thiol groups may be used, as described, for example, in U.S. Pat. No. 5,412,076 to Flamel Technologies, the disclosure of which is incorporated herein. Proteinaceous matrices including collagen also are described in U.S. Pat. No. 4,619,913 to Matrix Pharmaceuticals, the disclosure of which is incorporated herein.

Drug delivery systems based on hyalurans, for example, including hyaluronan or hyaluronan copolymerized with a hydrophilic polymer or hylan, may be used, as described in U.S. Pat. No. 5,128,326 to Biomatrix Inc., the disclosure of which is incorporated herein.

Hydrogel materials available in the art may be used. Exemplary materials include poly(hydroxyethyl methacrylate) (poly(HEMA)), water-insoluble polyacrylates, and agarose, polyamino acids such as alginate and poly(L-lysine), poly(ethylene oxide) (PEO) containing polymers, and polyethylene glycol (PEG) diacrylates. Other examples of hydrogels include crosslinked polymeric chains of methoxy poly(ethylene glycol) monomethacrylate having variable lengths of the polyoxyethylene side chains, as described in Nagaoka, et al., in Polymers as Biomaterials (Shalaby, S. W., et al., Eds.), Plenum Press, 1983, p. 381, the disclosures of which are incorporated herein.

Hydrogels may be used that include hydrophilic and hydrophobic polymeric components in block (as disclosed in Okano, et al., J. Biomed. Mat. Research, 15, 393, 1981), or graft copolymeric structures (as disclosed in Onishi, et al., in Contemporary Topics in Polymer Science, (W. J. Bailey & T. Tsuruta, eds.), Plenum Publ. Co., New York, 1984, p. 149), and blends (as disclosed in Shah, Polymer, 28, 1212,1987; and U.S. Pat. No. 4,369,229), and the disclosures of each of these citations is incorporated herein by reference.

Hydrogels comprising acrylic-terminated, water-soluble chains of polyether dl-polylactide block copolymers may be used. Hydrogels may comprise polyethylene glycol, a poly(α-hydroxy acid), such as poly(glycolic acid), poly(DL-lactic acid) or poly(L-lactic acid) and copolymers thereof, or poly(caprolactone) or copolymers thereof. In one embodiment, the hydrogel may comprise a copolymer of poly(lactic acid) and poly(glycolic acid), also referred to herein as a poly-lactide-co-glycolide (PLGA) polymer. Hydrogels may be used that are polymerized and crosslinked macromers, wherein the macromers comprise hydrophilic oligomers having biodegradable monomeric or oligomeric extensions, terminated on the free ends thereof with end cap monomers or oligomers capable of polymerization and cross linking. The hydrophilic core itself may be degradable, thus combining the core and extension functions. The macromers are polymerized for example using free radical initiators under the influence of long wavelength ultraviolet light, visible light excitation or thermal energy. Biodegradation occurs at the linkages within the extension oligomers and results in fragments which are non-toxic and easily removed from the body. Exemplary hydrogels are described in U.S. Pat. Nos. 5,410,016, 5,626,863 and 5,468,505, the disclosures of which are incorporated herein.

Hydrogels based on covalently crosslinked networks comprising polypeptide or polyester components as the enzymatically or hydrolytically labile components may be used as described in Jarrett, et al., Trans. Soc. Biomater., Vol. XVIII, 182, 1995; Pathak, et al., Macromolecules., 26, 581, 1993; Park, et al., Biodegradable Hydrogels for Drug Delivery, Technomic Publishing Co., Lancaster, Pa., 1993; Park, Biomaterials, 9, 435, 1988; and W. Shalaby, et al., 1992, the disclosures of which are incorporated herein. Hyaluronic acid gels and polyhydroxyethylmethacrylate gels may be used.

Additionally, the delivery matrix may include a targeting ligand which permits targeted delivery of the angiogenic factor to a preselected site in the body. The targeting ligand is a specific binding moiety which is capable of binding specifically to a site in the body. For example, the targeting ligand may be an antibody or fragment thereof, a receptor ligand, or adhesion molecule selective or specific to the desired target site. Examples of target sites include vascular intercellular adhesion molecules (ICAMs), and endothelial cell-surface receptors, such as αᵥβ₃. Embodiments of delivery matrices including a targetting ligand include antibody-conjugated liposomes, wherein the antibody is linked to the liposome via an avidin-biotin linker, which are described, for example, in Sipkins, Radiology, 197:276 (1995) (Abstract); and Sipkins et al., Radiology 197:129 (1995) (Abstract).

Formulations and Methods of Administration

The angiogenic factor, optionally in a carrier, or formulation thereof, may be administered by a variety of routes known in the art including topical, oral, parenteral (including intravenous, intraperitoneal, intramuscular and subcutaneous injection as well as intranasal or inhalation administration) and implantation. The delivery may be systemic, regional, or local. Additionally, the delivery may be intrathecal, e.g., for CNS delivery. For example, administration of the angiogenic factor for the treatment of wounds may be by topical application of the angiogenic factor to the wound, systemic administration by enteral or parenteral routes, or local or regional injection or implantation. The angiogenic factor may be formulated into appropriate forms for different routes of administration as described in the art, for example, in “Remington: The Science and Practice of Pharmacy”, Mack Publishing Company, Pennsylvania, 1995, the disclosure of which is incorporated herein by reference.

The angiogenic factor, optionally incorporated in a controlled release matrix, may be provided in a variety of formulations including solutions, emulsions, suspensions, powders, tablets and gels. The formulations may include excipients available in the art, such as diluents, solvents, buffers, solubilizers, suspending agents, viscosity controlling agents, binders, lubricants, surfactants, preservatives and stabilizers. The formulations may include bulking agents, chelating agents, and antioxidants. Where parenteral formulations are used, the formulation may additionally or alternately include sugars, amino acids, or electrolytes.

Excipients include polyols, for example of a molecular weight less than about 70,000 kD, such as trehalose, mannitol, and polyethylene glycol. See for example, U.S. Pat. No. 5,589,167, the disclosure of which is incorporated herein. Exemplary surfactants include nonionic surfactants, such as Tween® surfactants, polysorbates, such as polysorbate 20 or 80, etc., and the poloxamers, such as poloxamer 184 or 188, Pluronic(r) polyols, and other ethylene/polypropylene block polymers, etc. Buffers include Tris, citrate, succinate, acetate, or histidine buffers. Preservatives include phenol, benzyl alcohol, metacresol, methyl paraben, propyl paraben, benzalconium chloride, and benzethonium chloride. Other additives include carboxymethylcellulose, dextran, and gelatin. Stabilizing agents include heparin, pentosan polysulfate and other heparinoids, and divalent cations such as magnesium and zinc.

The angiogenic factor, optionally in combination with a controlled delivery matrix, may be processed into a variety of forms including microspheres, microcapsules, microparticles, films, and coatings. Methods available in the art for processing drugs into polymeric carriers may be used such as spray drying, precipitation, and crystallization. Other methods include molding techniques including solvent casting, compression molding, hot-melt microencapsulation, and solvent removal microencapsulation, as described, for example in Laurencin et al., “Poly(anhydrides)” in “Biomedical Applications of Synthetic Biodegradable Polymers”, J. Hollinger, Ed., CRC Press, Boca Raton, 1995, pp. 59-102, the disclosure of which is incorporated herein.

In one embodiment, it is advantageous to deliver the angiogenic factor locally in a controlled release carrier, such that the location and time of delivery are controlled. Local delivery can be, for example, to selected sites of tissue, such as a wound or other area in need of treatment, or an area of inadequate blood flow (ischemia) in tissue, such as ischemic heart tissue or other muscle such as peripheral.

The angiogenic factor, optionally in combination with a carrier, such as a controlled release matrix, also may be administered locally near existing vasculature in proximity to an ischemic area for an indication such as an occlusive vascular disease, to promote angiogenesis near the area being treated.

Nucleic Acid Therapy

The angiogenic factor also may be administered by administering a nucleic acid encoding for the angiogenic factor. Nucleic acid polymers encoding angiogenic factors thus may be administered therapeutically. Nucleic acid polymers (DNA or RNA) encoding angiogenic factors are incorporated into nucleic acid constructs (gene transfer vectors), which include the appropriate signals (e.g., enhancers, promoters, intron processing signals, stop signals, poly-A addition sites, etc.) for the production of the angiogenic factor in the cells of the patient. The angiogenic factor-encoding nucleic acid constructs may be delivered systemically, regionally, locally, or topically, preferably delivered topically, locally or regionally, to induce production of the angiogenic factors by cells of the patient's body. Alternately, the angiogenic factor-encoding nucleic acid constructs may be delivered to a remote site, which will produce angiogenic factor and allow for its dispersal throughout the patient's body.

The angiogenic factor-encoding nucleic acid constructs may be delivered as “naked DNA” (i.e., without any encapsulating membrane or viral capsid/envelope). Muscle cells, particularly skeletal muscle cells as well as cardiac muscle cells are known to take up naked DNA and to express genes encoded on the naked DNA. This method of delivering a angiogenic factor-encoding nucleic acid construct is one preferred mode for the treatment of coronary artery disease. The naked DNA comprising a angiogenic factor-encoding nucleic acid construct can be locally delivered, e.g., by injection into cardiac muscle in areas surrounding a blockage, in lieu of or in conjunction with surgical treatment for the blockage. DNA vehicles for nonviral gene delivery using a supercoiled minicircle also may be used, as described in Darquet et al., Gene Ther., 4:1341-1349 (1997), the disclosure of which is incorporated herein.

Angiogenic factor-encoding nucleic acid constructs may also be delivered in non-cellular delivery systems, such as liposomes, or cationic lipid suspensions. The use of liposomes for gene transfer therapy is well known (see, for example, Lee et al., Crit. Rev. Ther. Drug Carrier Syst., 14(2):173-206 (1997); Lee and Huang, Crit Rev Ther Drug Carrier Syst, 14:173-206 (1997) and Mahoto et al., Pharm. Res. 14:853-859 (1997), the disclosures of which are incorporated herein. Generally, the angiogenic factor-encoding nucleic acid constructs are incorporated into or complexed with liposomes which may be further derivatized to include targeting moieties, such as antibodies, receptor ligands, or adhesion molecules selective or specific to the desired target site. The liposome systems for the delivery of angiogenic factor-encoding nucleic acid constructs may include DNA/cationic liposome complexes, neutral or anionic liposomes which encapsulate the constructs, polycation-condensed DNA entrapped in liposomes, or other liposome systems known in the art.

Carrier proteins that facilitate target cell specific gene transfer via receptor mediated endocytosis may be used as described in Uherek et al., J. Biol. Chem., 273:8835-8841 (1998). Glycosylated poly(amino acids) also are useful nonviral vectors for gene transfer into cells as described in Kollen, Chest, 111:95S-96S (1997), the disclosure of which is incorporated herein. Gene transfer may also be implemented by biolistic processes, such as jet injection as described in Furth, Mol. Biotech., 7:139-143 (1997), the disclosure of which is incorporated herein. Nonviral methods of gene transfer which may be used, such as gene gun, electroporation, receptor-mediated transfer, and artificial macromolecular complexes are described in Zhdanov et al., Vopr Med Khim, 43:3-12 (1997), the disclosure of which is incorporated herein. DNA may be complexed to protein, lipid, peptide, or other polymeric carriers with tissue targeting ligands as described in Sochanik et al., Acta Biochim Pol 43:293-300 (1996), the disclosure of which is incorporated herein. The use of glycotargeting, using ligands to lectins that are then endocytosed is described in Wadhwa et al., J. Drug Target. 3:111-127 (1995), and Phillips, Biologicals, 23:13-16 (1995), the disclosures of which are incorporated herein.

Viral vectors incorporating angiogenic factor-encoding nucleic acid constructs are also useful for delivery. The use of viral constructs for gene therapy is well known (see Robbins et al., Trends Biotechnol. 16(1):35-40 (1998) for a review). Viruses useful for gene transfer include retroviruses (particularly mouse leukemia virus, MLV, mouse mammary tumor virus, MMTV, and human endogenous retrovirus), adenoviruses, herpes-simplex viruses and adeno-associated viruses. The viral vectors useful for gene transfer according to the instant invention may be replication competent or incompetent. Replication incompetent viral vectors are currently preferred for retroviral vectors. Generally, the angiogenic factor-encoding nucleic acid construct is incorporated into a vector which includes sufficient information to be packaged, frequently by a specialized packaging cell line, into a viral particle. If the viral vector is replication competent, the viral vector will also include sufficient information to encode the factors and signals required for replication of new infectious viral particles. Viral particles incorporating the angiogenic factor-encoding nucleic acid constructs are injected or infused into or applied to the desired site.

Production of Angiogenic Factors

In one embodiment, angiogenic factors may be produced recombinantly using any of a variety of methods available in the art. For those angiogenic factors which are not glycosylated and for those angiogenic factors where glycosylation is not required for the activity of the factor (e.g., FGF-1 and FGF-2), the angiogenic factor may be produced by purification from natural sources or by recombinant expression in prokaryotic or eukaryotic host cells. For those angiogenic factors where glycosylation is required or desired for activity, purification from natural sources or recombinant production in eukaryotic host cells is appropriate. Angiogenic factors for use in the instant invention are preferably produced by recombinant expression and are purified.

The exact manner and protocol for purification of angiogenic factors from natural sources will depend on the source material and the particular angiogenic factor, as is well known in the art. Purification methods for angiogenic factors have been published and may be easily replicated.

For recombinant production, a DNA molecule encoding the protein is incorporated into an “expression construct” which contains the appropriate DNA sequences to direct expression in the recombinant host cell. Construction of expression constructs is well known in the art, and variations are simply a matter of preference.

Human, bovine and rat cDNAs encoding pleiotrophin have been sequenced. Fang et al., J. Biol. Chem., 267:25889-25897 (1992); Li et al. (1990) supra; Lai et al. (1992), supra; Kadomatsu et al., Biochem. Biophys. Res. Commun. 151:1312-1318 (1988); Tomomura et al., J. Biol. Chem. 265:10765-10770 (1990); Vrios et al., Biochem. Biophys. Res. Commun. 175:617-624 (1991); and Li et al., J. Biol. Chem., 267:26011-26016 (1992). However, there are a number of splice variants which can produce different isoforms of the protein. In one preferred isoform isolated from human sources, the mature protein is 136 amino acids (e.g., the protein encoded by bases 573-980 of SEQ ID NO 1), which is produced by proteolytic cleavage of a 32 amino acid N-terminal signal sequence from the 168 amino acid proprotein (e.g., the protein encoded by bases 477-980 of SEQ ID NO 1).

Human, mouse, chicken and Xenopus laevis cDNAs for midkine have also been sequenced (Tsutsui et al., Bioch. Biophys. Res. Comm., 176(2):792-797 (1991); Fu et al., Gene, 146(2):311-312; and Urios et al., Bioch. Biophys. Res. Comm., 175:617-624 (1991)). Alternate mRNAs for midkine have been detected, although the variation appears to be in the 5′ untranslated region (5′-UTR) of the mRNAs. A preferred midkine protein from human sources is the 121 amino acid mature protein, which is a product of proteolytic processing of the 143 amino acid precursor protein (see, for example, the protein and nucleotide sequences disclosed in Genbank accession no. M69148).

Human cDNAs for a number of different members of the VEGF family have been cloned and sequenced, including VEGF (Weindel et al., Biochem. Biophys. Res. Comm. 183(3):1167-1174 (1992)), VEGF 2 (Hu et al., International Patent Application No. WO 95/24473), VEGF-C (Joukov et al., EMBO J. 15(2):290-298 (1996)) and VEGF-D (Yamada et al., Genomics 42(3):483-488 (1997), and the VEGF related factors, VRF186 and VRF167 (Grimmond et al., Genome Res. 6(2)122-129 (1996)).

Known cDNA sequences for the FGF family include FGF-1, also known as acidic FGF or aFGF (Yu et al., J. Exp. Med. 175(4):1073-1080 (1992)), FGF-2, also known as basic FGF or bFGF (Satoshi et al., Japanese patent application no. JP 1993262798), FGF-5 (Haub et al., Proc. Natl. Acad. Sci. U.S.A. 87:(20):8022-8026(1990)), FGF-6, also known as HST-2 (Iida et al., Oncogene 7(2):303-309(1992)), FGF-8 (Payson et al., Oncogene 13(1):47-53 (1996)), FGF-9 (Miyamoto et al., Mol. Cell. Biol. 13(7):4251-4259 (1993)), and FGF-10 (Emoto et al., J. Biol. Chem. 272(37)23191-23194 (1997)).

At least three members of the epidermal growth factor family (EGF) are known, and nucleic acid sequences are available for EGF (Bell et al., Nucleic Acids Res. 14(21):8427-8446 (1986)), transforming growth factor alpha (TGF-α, Jakowlew et al., Mol. Endocrinol. 2(11):1056-1063 (1988)) and TGF-αHIII (International Patent Application No. WO 97/25349).

Genes encoding for the PDGFs are also known. mRNAs coding for the A and B chains have been cloned and sequenced, allowing recombinant production (Betsholtz et al., Nature 320(6064):695-699 (1986); and Collins et al., Nature 316(6030):748-750 (1985)).

A large number of methods are known for the production of proteins in prokaryotic host cells. Normally, only the mature portion (i.e., that portion of the angiogenic factor which remains after normal post-translational processing is completed) of the angiogenic factor is used for expression in prokaryotes. The angiogenic factors may be expressed “directly” (i.e., the angiogenic factor is produced without any fusion or accessory sequences) or as a fusion protein. Direct expression of angiogenic factors in prokaryotic host cells will normally result in the accumulation of ‘refractile’ or ‘inclusion’ bodies which contain the recombinantly expressed protein. The inclusion bodies can be collected, then resolubilized. Angiogenic factors produced in inclusion bodies will normally require “refolding” (i.e., resolubilization and reduction followed by oxidation under conditions which allow the protein to assume its native, properly-folded conformation) to regenerate biologically active protein. Refolding protocols are well known in the art, and there are several refolding methods which are considered to be generally applicable to all proteins (see, for example, U.S. Pat. Nos. 4,511,502, 4,511,503, and 4,512,922). Refolded angiogenic proteins may be conveniently purified according to any of the methods known in the art, particularly by use of the protocols developed for the purification of the factors from natural sources.

There are a vast number of possible fusion partners for the angiogenic factor if the factor is expressed as a fusion protein in prokaryotic host cells. Fusion proteins containing leader sequences from periplasmic proteins are secreted into the periplasm of gram negative bacteria such as E. coli. The leader sequence is frequently cleaved upon secretion into the periplasmic space, resulting in production of the angiogenic factor without any N-terminal extension sequences. Advantageously, many mammalian proteins fold into their native, active conformation when expressed in the periplasmic space, due to the presence of “chaperone” proteins and the more oxidizing environment of the periplasm. Fusion proteins may also be made with amino acid sequences which maintain the solubility of the expressed fusion protein or with amino acid sequences which act as a “tag” (i.e., a sequence which can be used to easily identify or purify the fusion protein) such as oligo-histidine or a sequence which is a substrate for biotinylation by bacterial cells. Fusion proteins which are not naturally appropriately cleaved may also contain a protease recognition site which will allow the removal of the fusion partner sequence. Such sequences are well known in the art. Angiogenic factors produced as fusion proteins may require refolding, as noted above. After refolding, the angiogenic factor may be further purified according to any of the methods known in the art, particularly by use of the protocols developed for the purification of the factors from natural sources.

Recombinant production of proteins in eukaryotic cells is well known. Angiogenic factors may be produced in any eukaryotic host cell, including, but not limited to, budding or fission yeast, insect cells such as D. melanogaster cell lines, mammalian cell lines and plants. If the host cell is a host cell that recognizes and appropriately cleaves human signal sequences (e.g., mammalian cell lines), then the entire coding region of the angiogenic factor may be incorporated into the expression construct, otherwise only the portion encoding the mature protein is used. Expression constructs for use in eukaryotic host cells are well known in the art. Preferred systems for production of angiogenic factors include tobacco plant/tobacco mosaic virus systems, baculovirus/insect cell systems and mammalian cell lines. In the case where the angiogenic factor is pleiotrophin and it is expressed in mammalian cell lines, it is preferred that the expression construct contain the open reading frame (ORF) of pleiotrophin linked to heterologous 5′- and 3′-sequences, as the native 5′- and 3′-sequences may form antisense complexes with mRNAs encoding human proteins such as hsp 70.

In addition to recombinant production, angiogenic factors also may be produced synthetically. For example, peptides, including peptide fragments of naturally occurring growth factors, with angiogenic activity, may be synthesized using solid phase techniques available in the art. Additionally, analogues, which act as growth factor mimics, may be synthesized using synthetic organic techniques available in the art, as described for example in: March, “Advanced Organic Chemistry”, John Wiley & Sons, New York, 1985. Analogues include small molecule peptide mimetics, as well as synthetic active peptides homologous to naturally occurring angiogenic factors or fragments thereof.

All references cited herein are hereby incorporated by reference in their entirety.

The invention will be understood by the following nonlimiting Examples.

### EXAMPLES

### Example 1

In Vitro Use of an Angiogenic Factor

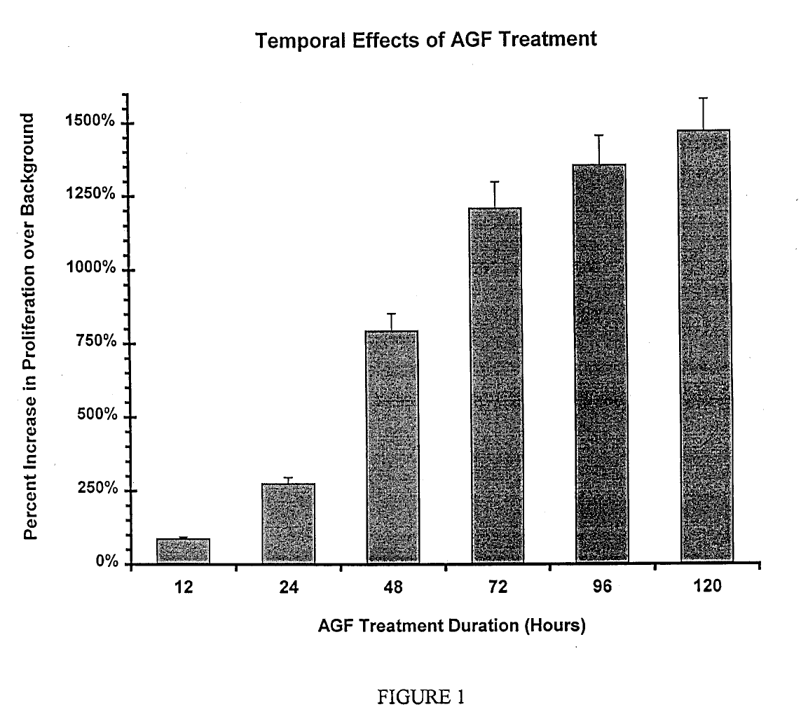

Recombinant human pleiotrophin (PTN) was isolated as described in Fang et al., J. Biol. Chem., 267:25889-25897 (1992)). To determine the percent increase in endothelial cell proliferation after PTN stimulation in vitro, endothelial cells (HUVEC, human umbilical vein endothelial cells, American Type Culture Collection, # CRL-1730) were seeded at 10⁴ cells per well into 12 well tissue culture plates, in 2 ml F12K media containing 10% fetal bovine serum (Life Technologies (Rockville Md.), # 11765054 and # 16140071, respectively) using standard cell culture procedures. After approximately six hours to allow the cells to become adherent to the culture plate, 50 ng PTN in 50 μl PBS buffer (phosphate buffered saline) was added to each treatment well (n=6 in each of six treatment groups). Equivalent volume of PBS only was added to each control well (n=6) to determine background proliferation level. Media was removed from the wells, cells washed twice with 2 ml PBS and 2 ml media replenished at each 24 hour time point, except for the 12 hour group which was replenished with media at 12 hours. The same dose PTN was also replenished at each 24 hour point up to the indicated treatment duration, after which media only was replenished. At the end of one week, cells were made disadherent and counted by standard cell culture technique. FIG. 1 shows the average percent increase in each treatment group after subtracting out average background (untreated) proliferation.

### Example 2

Treatment of a Mouse Wound with an Angiogenic Factor In Vivo

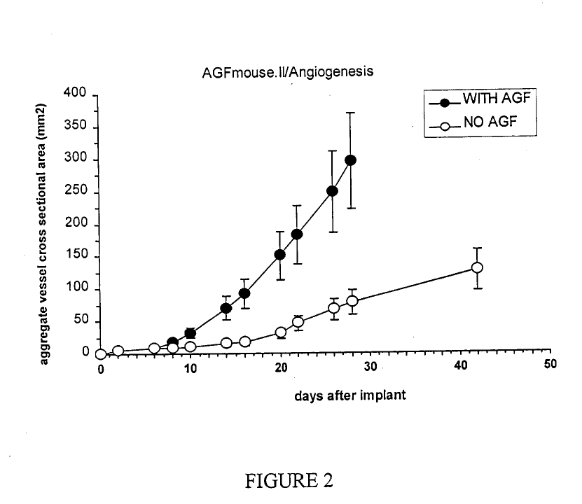

PTN was isolated as described in Example 1. To determine the effects of local PIN treatment in vivo on the subcutaneous vasculature in mice, matrix implants were injected-bilaterally under the loose flank skin of BALB/c mice (Harlan Sprague-Dawley, Indianapolis, Ind.), five mice per group (n=10). To make implants, PTN protein in PBS solution (as above) was mixed into Matrigel™ (Collaborative Research, Mass.), a liquid at room temperature, at a concentration of 10 μg/ml. Control implants were made similarly, but without PTN in the buffer. As matrix solution began to gel as temperature was increased to above ambient temperature, but below body temperature of 37° C., volumes of 1 ml per site were injected into a subdermal pouch using a 16 gauge needle. The gel solution became a partially solid matrix at body temperature. At each time point, the respective group of mice was sacrificed and the overall density and diameters of landmark vessels in the region of the implant were measured using standard microcalipers. FIG. 2 shows the average aggregate vessel size between the treated (+PTN) and untreated (−PTN) groups over time.

### Example 3

In Vivo Angiogenesis Using a Controlled Delivery Matrix

PTN was obtained as described in Example 1. To determine the effects of sustained local PTN treatment on a functional vascular system in vivo, the well known Folkman CAM (chicken chorioallantoic membrane) assay was used. After partially opening the egg shells of five day old fertilized chicken eggs (local Leghorn white, Half Moon Bay, Calif.), a Vasotrophin™ system (Angiogenix Inc, Burlingame, Calif.) was placed on the leading edge of the CAM, which was approximately 15 mm diameter. The Vasotrophin™ system used was a 500 μl bioerodible pellet consisting of PTN formulated into a matrix of poly(lactide-co-glycolide) (PLGA, Absorbable Polymer Technologies, Birmingham, Ala.) at 1 μg/ml, or each containing 500 ng PTN. The control pellets were produced similarly, but without PTN. CAMs were visualized over the next two weeks and the differences in blood vessel growth patterns were observed and imaged through a dissecting microscope camera.

The blood vessels in the vicinity of the growth factor-containing Vasotrophin systems demonstrated a marked increase in both vessel density and caliber. There was also radial ingrowth, or directional growth of vessels toward the pellets. In the control CAMs, the blood vessels continued to grow in the same manner as the completely untreated CAM, in which nothing was placed on the membrane. The control vessels were significantly less dense and smaller in diameter; they also grew directionally without regard to the pellets. This demonstrates the direct and specific stimulation of increased vessel density and caliber upon sustained local exposure to PTN.

What is claimed is:

1. A method of stimulating angiogenesis in a human or animal in need thereof, the method comprising administering to the human or other animal a therapeutically effective amount of a pleiotrophin or midkine molecule in a pharmaceutically acceptable carrier.

2. The method of claim 1, wherein the pleiotrophin or midkine molecule is a pleiotrophin or midkine protein.

3. The method of claim 1, wherein the carrier comprises a controlled release matrix that permits controlled release of the pleiotrophin or midkine molecule.

4. The method of claim 3, wherein the carrier comprises a ligand capable of targeting the pleiotrophin or midkine molecule to a preselected site in the body.

5. The method of claim 1, wherein the molecule is administered to the vascular system.

6. The method of claim 1, wherein the molecule is administered to the cardiovascular system.

7. The method of claim 6, wherein the molecule is administered in a therapeutically effective amount for the treatment of a condition selected from the group consisting of coronary artery disease and ischemic heart disease.

8. The method of claim 1, wherein the molecule is administered to the peripheral vascular system.

9. The method of claim 8, wherein the molecule is administered in a therapeutically effective amount for the treatment of a condition selected from the group consisting of diabetic peripheral vasculopathies and peripheral atherosclerotic disease.

10. The method of claim 1, wherein the molecule is administered locally in a therapeutically effective amount to a wound to promote wound healing.

11. The method of claim 10, wherein the wound is selected from the group consisting of an ulcer, a pressure sore, a surgically induced wound, and a traumatically induced wound.

12. The method of claim 1, wherein the molecule is administered locally in a therapeutically effective amount to tissue comprising nerves to treat a neurological condition.

13. The method of claim 12, wherein the molecule is administered in a therapeutically effective amount for the treatment of a condition selected from the group consisting of stroke, multi-infarct dementia, and general brain ischemia.

14. The method of claim 1, wherein the molecule is administered locally in a therapeutically effective amount to tissue comprising bone or cartilage.

15. The method of claim 14, wherein the molecule is administered in a therapeutically effective amount for the treatment of a condition selected from the group consisting of osteoporosis, arthritis and joint replacement or repair.

16. The method of claim 1, wherein the molecule is a pleiotrophin protein.

17. The method of claim 1, wherein the molecule is a pleiotrophin molecule, and wherein the pleiotrophin molecule is a pleotrophin protein isolated from a human cell source, or an active fragment or analogue thereof.

18. The method of claim 16, wherein the protein is produced recombinantly in a eukaryotic host cell.

19. The method of claim 1, wherein the molecule is a midkine molecule, and wherein the midkine molecule is a midkine protein isolated from a human or animal cell source, or an active fragment or analogue thereof.

20. The method of claim 3, wherein the controlled release matrix comprises a polymer.

21. The method of claim 20, wherein the polymer comprises a biodegradable or bioerodable polymer.

22. The method of claim 20, wherein the polymer is selected from the group consisting of poly(esters), poly(anhydrides), and poly(amino acids).

23. The method of claim 20, wherein the polymer is a silk elastin poly(amino acid) block copolymer.

24. The method of claim 1, wherein the carrier comprises a liposome.

25. The method of claim 24, wherein liposome comprises a targeting ligand capable of targeting the liposome to a preselected site in the body.

26. The method of claim 1, wherein the molecule is administered locally in a therapeutically effective amount to an organ transplant site to promote engraftment of the transplant in the host.

27. A method of stimulating angiogenesis in a human or animal in need thereof, the method comprising administering to the human or animal a therapeutically effective amount of an angiogenic factor in a pharmaceutically acceptable carrier comprising a silk elastin poly(amino acid) block copolymer.

28. The method of claim 27, wherein the angiogenic factor is selected from the group consisting of pleiotrophin, midkine, fibroblast growth factor (FGF) family members, vascular endothelial growth factor (VEGF) family members, platelet derived growth factors, and epithelial growth factor (EGF) family members.

29. A method of stimulating angiogenesis in a human or animal in need thereof, the method comprising administering to the human or animal a therapeutically effective amount of an angiogenic factor in a pharmaceutically acceptable carrier comprising poly-lactide-co-glycolide;
wherein the angiogenic factor is selected from the group consisting of a pleiotrophin and midkine molecule.

30. A pharmaceutically acceptable composition for the therapeutic delivery of a pleiotrophin or midkine molecule to a human or animal, the composition comprising a pleiotrophin or midkine molecule and a pharmaceutically acceptable carrier.

31. The composition of claim 30, wherein the pleiotrophin or midkine molecule is a pleiotrophin or midkine protein.

32. The composition of claim 30, wherein the carrier comprises a polymer capable of controlled release of the molecule.

33. The composition of claim 32, wherein the polymer is selected from the group consisting of poly(esters), poly(anhydrides), and poly(amino acids).

34. The composition of claim 32, wherein the polymer is biodegradable or bioerodible.

35. The composition of claim 32, wherein the polymer is a silk elastin poly(amino acid) block copolymer.

36. The composition of claim 30, wherein the carrier comprises a liposome.

37. The composition of claim 36, wherein the carrier comprises a liposome comprising a targeting ligand capable of targeting the liposome to a preselected site in the body.

38. The composition of claim 36, wherein the liposome comprises a heterovesicular liposome.

39. The composition of claim 30, wherein the molecule is a pleiotrophin molecule.

40. The composition of claim 39, wherein the pleiotrophin molecule is a pleiotrophin protein isolated from a human cell source, or an active fragment or analogue thereof.

41. The composition of claim 30, wherein the molecule is a midkine protein.

42. A method for stimulating angiogenesis in a human or animal in need thereof, the method comprising administering to the human or animal a therapeutically effective amount of a gene transfer vector encoding the production of a pleiotrophin or midkine protein in a pharmaceutically acceptable carrier.

43. The method of claim 42, wherein the gene transfer vector encodes the production of a pleiotrophin protein.

44. The method of claim 42, wherein the gene transfer vector encodes the production of a midkine protein.

45. The method of claim 43, wherein the gene transfer vector is naked DNA.

46. The method of claim 43, wherein the method comprises administering the gene transfer vector in combination with liposomes.

47. The method of claim 43, wherein the gene transfer vector is a viral vector.

48. The method of claim 44, wherein the gene transfer vector is naked DNA.

49. The method of claim 44, wherein the method comprises administering the gene transfer vector in combination with liposomes.

50. The method of claim 44, wherein said gene transfer vector is a viral vector.


SEQUENCE LISTING (1 sequence)

SEQ ID NO: 1 - DNA, 1383 bp; single-stranded, linear
aagtaaataaactttaaaaatggcctgagttaagtgtattaaaaagaagaaatagtcgtaagatggcagtataaattcatctctgcttttaataagcttcccaatcagctctcgagtgcaaagcgctctccctccctcgcccagccttcgtcctcctggcccgctcctctcatccctcccattctccatttcccttccgttccctccctgtcagggcgtaattgagtcaaaggcaggatcaggttccccgccttccagtccaaaaatcccgccaagagagccccagagcagaggaaaatccaaagtggagagaggggaagaaagagaccagtgagtcatccgtccagaaggcggggagagcagcagcggcccaagcaggagctgcagcgagccgggtacctggactcagcggtagcaacctcgccccttgcaacaaaggcagactgagcgccagagaggacgtttccaactcaaaaatgcaggctcaacagtaccagcagcagcgtcgaaaatttgcagctgccttcttggcattcattttcatactggcagctgtggatactgctgaagcagggaagaaagagaaaccagaaaaaaaagtgaagaagtctgactgtggagaatggcagtggagtgtgtgtgtgcccaccagtggagactgtgggctgggcacacgggagggcactcggactggagctgagtgcaagcaaaccatgaagacccagagatgtaagatcccctgcaactggaagaagcaatttggcgcggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaagcagaatctaagaagaagaaaaaggaaggcaagaaacaggagaagatgctggattaaaagatgtcacctgtggaacataaaaaggacatcagcaaacaggatcagttaactattgcatttatatgtaccgtaggctttgtattcaaaaattatctatagctaagtacacaataagcaaaaacaaccaatttgggttctgcaggtacatagaagttgccagcttttcttgccatcctcgccattcgaatttcagttctgtacatctgcctatattccttgtgatagtgctttgctttttcatagataagcttcctccttgcctttcgaagcatcttttgggcaaacttctttctcaggcgcttgatcttcagctctgcgaaattccttcgctttttcttaagggtttctggcacagcaggaacctccttcttcttctcttctacaccctctatgtacc